(19) United States
(12) Patent Application Publication
(10) Pub. No.: US 2019/0253748 A1
(43) Pub. Date: Aug. 15, 2019

(54) SYSTEM AND METHOD OF MIXING AND SYNCHRONISING CONTENT GENERATED BY SEPARATE DEVICES

(71) Applicant: Stephen P. Forte, Beverly Hills, CA (US)

(72) Inventor: Stephen P. Forte, Beverly Hills, CA (US)

(21) Appl. No.: 15/676,627

(22) Filed: Aug. 14, 2017

(51) Int. Cl. H04N 21/43; H04N 7/18; A61B 5/0205

(52) U.S. Cl. CPC H04N 21/4307; A61B 5/0205; H04N 7/181

(57) ABSTRACT

A system for combining separately recorded content is provided. The system comprises a mobile device and an application stored in the mobile device that when executed receives entry of a first instruction and causes, based on the receipt of the first instruction, a remote video camera to activate and record video content. The system also activates, based on the receipt of the first instruction, an audio recorder resident on the mobile device, receives entry of a second instruction, and terminates, based on the receipt of the second instruction, the video recording and audio recording. The system also downloads the recorded video content to the mobile device, or the audio to the video device, and mixes the recorded video content and audio content into a single video file comprised of at least the audio and video recordings. The mixed content is stored in a common video file format.

### CROSS REFERENCE TO RELATED APPLICATIONS

This application is a continuation of U.S. provisional patent application No. 62/532,207, filed Jul. 13, 2017.

### FIELD OF THE DISCLOSURE

The present disclosure is in the field of wireless devices. More particularly, the present disclosure is in the technical field of mixing video content and audio content recorded simultaneously or non-simultaneously on separate devices and creating a single viewable presentation therefrom.

### BACKGROUND OF THE DISCLOSURE

Users of action video cameras may wish to clearly narrate, on a real time and concurrent basis, the video content they are capturing as they engage in vigorous and possibly extreme or even dangerous physical activity. They may wish to record clear and intelligible voice or other audio content while engaging in the activity as opposed to producing a voice narrative once recording of video has concluded, or using a musical score. Such users may want to have their video and audio content recorded simultaneously, to then to be combined and synchronized into a single file via a simple action. The users may wish to have such combining of content to be done immediately after the separate content has been recorded or after. Such users may then view and listen to the combined content shortly after the experience, send the recording to friends, or post the recording on a public venue.

### SUMMARY OF THE DISCLOSURE

Systems and methods described herein provide a mobile device and an application executing thereon that receives video content and audio content simultaneously recorded on separate devices and mixes the content to create a single synchronized presentation providing both items of content. The video camera may be distant from the mobile device and be unable to record audio or only able to record audio compromised by significant background or environmental noise that renders voice audio of poor quality. The mobile device instead records audio spoken by the user or target of the video, possibly by a microphone proximate the user's mouth or using a distant shotgun mic.

After the mobile device transmits instructions causing the video camera and audio recording to end, the recorded video content is received by the mobile device. The application provided herein mixes the received video content with the recorded audio content in a synchronized manner to create a single presentation.

Systems and methods also provide for telemetrically gathered data to also be added to the mixed video and audio content. Biometric data such as heart rate, environmental data such as temperature or wind speed, or, for example, speed of a vehicle ridden by a user may be observed by sensors and transmitted to the mobile device which then mixes and presents the telemetrically gathered data in text or other viewable format in the presentation.

The video camera may be body-worn by the user, and may for example be mounted to a helmet of the user. The camera may not be attached to the user and may instead be stationary or may be mounted to a moving object separate from the user, for example on a drone device.

### BRIEF DESCRIPTION OF THE DRAWINGS

FIG. 1 is a block diagram of a system for mixing and synchronizing content generated by separate devices in accordance with an embodiment of the present disclosure.

### DETAILED DESCRIPTION OF THE INVENTION

Systems and methods described herein provide for receiving video content and audio content recorded on separate devices and mixing the video and audio material on a local device, thus enabling the mixed content to be immediately played back as a single, synchronized media presentation. An application is provided herein that executes on a mobile device, for example an iPhone or on a smart camera. The mobile device receives video content recorded by a separate device, for example an action camera that may be mounted on a user's helmet, surfboard, or skis. The action camera may in embodiments not be near the user and may instead be mounted on another device or object that may be stationary or in motion, for example a drone device.

The mobile device records audio content simultaneous with the video camera recording action video. The audio content may be spoken by a user, for example into a microphone near the user's mouth and transmitted via wired or wireless connection to the mobile device. Upon the recording of the video content by the video camera being stopped, the recorded video content is transmitted to the mobile device. The application provided herein executing on the mobile device then mixes the received video content with the locally recorded audio content into a single playable narrated action video.

Systems and methods also provide for telemetrically sourced data to also be mixed and presented with recorded audio and video material. Sensors attached to and/or proximate the user may, for example, record the user's heart rate or speed of a bicycle the user is riding. Wind speed, temperature or other environmental data may also be recorded. This captured data may be mixed by the application with the coincidentally captured audio and video camera and displayed the during the presentation at the points where the data was captured. For example, at the point in the display of the video that a bicycle rider's course is shown as nearing an end of a long uphill climb, the rider's heart rate and pulse are both displayed as text in the video. During that same period, the rider's voice may be heard narrating, perhaps in a winded fashion, how tired he/she had become at this difficult stage of the long course.

The application provided herein receives the aforementioned video, audio and telemetric digital material from separate devices as described above. The material may be recorded by those proximate devices or from more remote recording or storage sources and then forwarded to the mobile device by the proximate devices for content mixing. When the application on the mobile device receives an instruction from the user, the application sends a signal to the video camera to discontinue its recording of video and save the recorded video in at least one file. The application causes the at least one video file to be transmitted from the video camera to the mobile device.

The application then mixes the received video content with the locally recorded audio content as described. The presentation created from the application mixing the separately recorded content may then be viewed and listened to by the mobile device user immediately after mixing is complete. The user may also send the presentation via email or other means to friends or others and post the presentation to a widely accessible location, perhaps an Internet web site or to a social networking venue. The presentation may be made viewable by authorized parties only or may be made publicly available.

As noted, the video camera need not be attached to the user's body or on a bicycle, vehicle, skis, surfboard, or any other implement or device ridden or controlled by the user. In embodiments, the video camera is attached to a drone device or other device separate from the user. The video camera may record video from the sky as the drone device moves about. The user may be able, from the ground, the view the video content as it is being recorded and may record narrative audio into the mobile device to accompany the video content. As in the other embodiments, once the video camera receives an instruction to stop recording, the video content may be received by the mobile device and mixed with the user's recorded audio content and even with telemetric data recorded during the drone's flight.

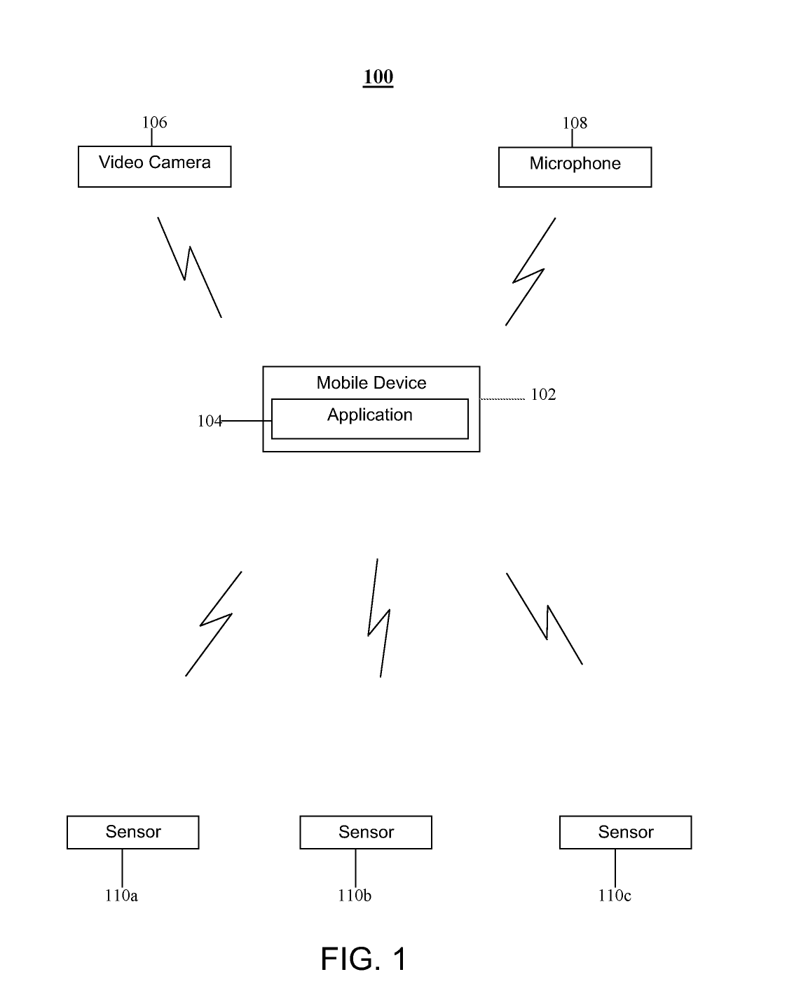

Turning to the figures, FIG. 1 is a block diagram of a system for mixing and synchronizing content generated by separate devices in accordance with an embodiment of the present disclosure. FIG. 1 depicts components of a system 100 provided herein. System 100 comprises a mobile device 102, an application 104, a video camera 106, a microphone 108, and a sensor 110. While the components of the system 100 are depicted as communicating with each other wirelessly, the components by communicate with each other via wired connection.

The mobile device 102 may be an iPhone® produced by Apple Inc. The application 104 executes on the mobile device 102 and supports the methods provided herein. The video camera 106 may be a small action camera provided by GoPro Inc. or other manufacturer. The application 104 receives instructions from a user of the mobile device to begin a session. The application 104 transmits an instruction to the video camera 106 to begin recording video content. The application 104 may simultaneously cause the mobile device 102 to begin recording audio content provided by the user via the microphone 108 that may be positioned near the user's mouth or in a remote location.

In some cases, the video camera 106 may be equipped with its own built-in microphone. However, the video camera 106 in many embodiments will be positioned not near the user's mouth and in some embodiments the video camera 106 may not be near the user at all. For example, the video camera 106 may be secured to a drone, balloon, kite or other aerial device. Because embodiments herein may involve the user engaging in action or extreme sports, for example bicycle touring, rock climbing, or hang gliding, wind and other background noise may be a factor. A microphone integrated into the video camera 106 might be too far from the user's mouth for the user's spoken narrative to be clearly recorded without being obscured by such background noise. The microphone 108 provided herein may be positioned close to the user's mouth with the user's spoken sounds captured there and sent directly to the mobile device 102 for recording and subsequent mixing with audio content received from the video camera 106.

As noted, the user may begin a session by providing an instruction to the application 104 executing on the mobile device 102. The instruction may be manually entered by the user, entered via a voice command, be received from a sensor triggered by the user or other party, or may be activated in another manner. Upon receiving such instruction, the application 104 transmits instructions to the video camera 106 to begin recording video content. The video camera 106 stores recorded video content locally.

At about the same time, the application 104 may activate the microphone 108 and begin recording audio content. The application 104 may track its signaling with the video camera 106 and the microphone 108 so that later synchronization of the received video content and the stored audio content may be performed more rapidly and with greater ease.

When the session is ended based on user instruction or based on occurrence of a predetermined event, the application 104 receives a message indicating that the recording of video content and audio content should be terminated. The application 104 transmits an instruction to the video camera 106 to stop recording and send saved video content to the mobile device 102. The mobile device 102 receives the video content from the video camera 106 and stores the video camera locally or elsewhere. The same is true for the recorded audio content. After recording of audio is terminated, the audio content may be stored locally or at another location.

The application 104 obtains the received video content and the audio content and synchronizes the content so that the audio content is audible by a viewer of the video content at the points within the video when the audio was recorded. Scenes within the video receive narration at the moments the scenes were recorded.

The application 104 mixes the synchronized video content and audio content to create a combined single presentation that may be invoked in that manner by activating an executable object. The mixed content may be stored in a common video file format. The actions of mixing the content and creating the presentation may be completed in moments after the recording is complete, allowing the user to view and listen to the presentation and enjoy it without having to wait for the content to be combined and otherwise processed by a desktop computer or professional-type operation that might have been previously required. At that moment, the user may also edit the combined content, send it to other parties via electronic mail or other means, and post the presentation on social media sites and electronic venues.

In an embodiment, the video content and audio content may be recorded at different times and mixed at a later date to produce the presentation. The audio content may be stored elsewhere and sent to the mobile device 102 at a later time for processing.

The sensor 110a may be used to gather telemetric data to add to the presentation. The sensor 110a may be body-worn by the user and may be a heart rate, pulse, respiratory or other biometric monitor. The data captured by the sensor 110a may be displayed in the presentation at the points in the presentation that the data was captured. The user's heart rate could be displayed at a key point in the presentation such as at a stressful point or along a group of points.

The sensor 110b may also capture non-biometric data related to the user's experience that is being captured in video and audio. The sensor 110b may be a speedometer attached to a bicycle that the user is riding or an altimeter attached to a hang glider the user is using.

The sensor 110c may also capture environmental data such as temperature, wind speed, or humidity. This data may be processed by the application 104 and displayed in the presentation superimposed as text over the video content as it is displayed.

The video camera 106 need not be attached to or even proximate the user. In an embodiment, the video camera 106 can be attached to a drone or other flying device with video content streamed on a near real time basis to the mobile device 102 on the ground. The user could watch the video stream and provide audio to accompany what the user sees in the video stream. The application 102 could later mix the audio content and video content as before.

In an embodiment, most or all of the functionality of the application is resident and executes on the video camera 106 instead of on the mobile device 102. In this embodiment, when the recording of the audio content and the video content are finished, instead of the video content being sent to the mobile device 102 for mixing there by the application 104 with the audio content recorded and stored there, the audio content is sent to the video camera 106 where the application 104 performs the mixing as described herein.

What is claimed is:

1. A system for combining separately recorded content, comprising:
a mobile device;
an application stored in the mobile device that when executed:
receives entry of a first instruction,
causes, based on the receipt of the first instruction, a remote video camera to activate and record video content,
activates, based on the receipt of the first instruction, an audio recorder resident on the mobile device,
receives entry of a second instruction,
terminates, based on the receipt of the second instruction, the video recording and audio recording,
downloads the recorded video content to the mobile device, and
mixes the recorded video content and audio content into a single video file comprised of at least the audio and video recordings

2. The system of claim 1, wherein the mixed content is stored in a common video file format.

3. The system of claim 1, wherein the instructions are received by the application at least one of via manual activation by a user and via electronic signal.

4. The system of claim 3, wherein the electronic signal is originated via user activation of at least one sensor.

5. The system of claim 1, wherein the mobile device is one of physically attached to a user and separate from the user.

6. The system of claim 1, wherein the remote video camera is not proximate the mobile device on the user's body.

7. The system of claim 1, wherein based on the receipt of the first instruction, an audio recorder is resident on the internet, with the audio passing through the mobile device.

9. The system of claim 1, wherein the audio and video files may be selected on the mobile device by the user to be mixed into a single file at any time after the recording is complete.

10. The system of claim 1, wherein the application alternately executes at least partially on the video camera.

11. A method of combining separately recorded content, comprising:
a mobile device causing a remote camera to begin recording video content;
the mobile device receiving data from at least one sensor;
the mobile device causing the camera to discontinue recording the video content;
the mobile device receiving the recorded video content; and
the mobile device mixing the data received from the at least one sensor with the received video content.

12. The method of claim 11, wherein the mixed data and video content are combined by the mobile device and thereafter playable as a single presentation.

13. The method of claim 11, wherein the data is telemetrically gathered and is displayed in at least text format during playback of the video content upon the presentation being invoked.

14. The method of claim 11, further comprising the mobile device recording audible sounds provided by at least a user of the mobile device coincident with the recording of the video content.

16. The method of claim 14, further comprising the mobile device mixing the audible sounds with the data received from the at least one sensor and the video content.

17. The method of claim 11, wherein the at least one sensor comprises at least one of a body-worn sensor gathering at least one of heart rate and respiratory data and a non-body-worn sensor reporting at least one of speed and performance data.

18. A system for combining separately recorded content, comprising:
a mobile device;
an application stored in the mobile device that when executed:
causes a remote video camera mounted on a drone device to begin recording video content,
displays the video content in near real time fashion,
records, simultaneous with the recording of the video content, audible input, the audible input associated with the recorded video content,
causes the camera to discontinue recording,
receives the recorded video content, and
mixes the recorded audible input with the video content recorded by the drone-mounted camera.

19. The system of claim 18, wherein the mixing of the audible input with the video content creates a single synchronized action video presentation.

20. The system of claim 18, wherein the application further mixes telemetric data received from the drone device with the audible input and the video content.
